(19) United States
(12) Patent Application Publication
(10) Pub. No.: US 2009/0062923 A1
(43) Pub. Date: Mar. 5, 2009

(54) Method and apparatus for shoulder arthroplasty

(76) Inventor: Todd V. Swanson, Las Vegas, NV (US)

Correspondence Address: WEIDE & MILLER, LTD., 7251 W. LAKE MEAD BLVD., SUITE 530, LAS VEGAS, NV, 89128, US

(21) Appl. No.: 11/899,473

(22) Filed: Sep. 5, 2007

(51) Int. Cl. A61F 2/40

(52) U.S. Cl. 623/19.13; 623/19.11; 623/19.14

(57) ABSTRACT

A prosthetic shoulder implant includes a humeral component having an anchor and a head, and a glenoid component. The glenoid component is defined by a body having an outer surface including one or more tissue and bone in-growing material areas, a concave inner surface for accepting the head of the humeral component, and defining a stop portion which prevents the head of the humeral component from moving upwardly into contact with the acromion or clavicle. A method of total shoulder arthroplasty includes locating the humeral and glenoid components so that the glenoid component prevents movement of the humeral component into contact with the acromion or clavicle, and such that the glenoid component is permanently anchored to the patient by tissue and bone ingrowth into the outer surface of the component.

### FIELD OF THE INVENTION

The present invention relates to methods and apparatus for shoulder replacement or arthroplasty.

### BACKGROUND OF THE INVENTION

For a variety of circumstances, shoulder replacement or arthroplasty may be necessary. For example, degenerative joint disorders such as rheumatoid arthritis and avascular necrosis, posttraumatic problems or injuries such as humeral head fractures, and dysplastic problems and tumors may all necessitate shoulder arthroplasty.

FIG. 1 illustrates a shoulder joint. In general, the shoulder is made up of three bones: the clavicle C, the humerus H and the scapula S. The rotator cuff connects the humerus H to the scapula S. The rotator cuff is formed by the tendons of four muscles: the supraspinatus, the infraspinatus, the teres minor, and the subscapularis. When normally configured, a head portion of the humerus H is located adjacent a glenoid portion G of the scapula S and is permitted to rotate relative to the scapula S and the clavicle C, as controlled by the rotator cuff.

Shoulder arthroplasty involves the placement of prosthetic shoulder implants. A variety of these implants are known. Generally, the implants include a glenoid component and a humeral component, these components configured to substitute for the head or ball of the humerus and the glenoid portion of the scapula of the normal shoulder.

FIG. 2 illustrates an embodiment of prosthetic shoulder implants in accordance with the prior art. As illustrated, the glenoid component GC is a slightly arcuate disc, having a front face and a rear face. One or more projections P extend from the rear face. Apertures are drilled in the scapula S and the projections of the glenoid component GC are inserted into those apertures in order to affix or anchor the glenoid component GC to the scapula S. So affixed, as best illustrated in FIG. 3, the front face of the glenoid component GC comprises a generally vertically extending or oriented face.

Referring again to FIG. 2, the humeral component HC comprises a head connected to a stem. After the humerus head is removed, the stem of the humeral component HC is extended into the top of the humerus H, generally along the central axis thereof. The head of the humeral component defines a curved front face. This curved front face of the head is configured to engage the front face of the glenoid component GC.

Use of these components has proven effective in total shoulder arthroplasty for addressing problems such as arthritic pain. These components and typical methods of shoulder arthroplasty are very often not effective, however, in the event of cuff tear arthropathy, large tears of the rotator cuff, or paralysis of the rotator cuff muscles. In such situations, the rotator cuff no longer effectively serves to maintain proper location of the humeral head relative to the glenoid, including during motion of the humerus (arm). In particular, during normal movement of the shoulder, some vertical movement of the humerus relative to the scapula and clavicle are permitted. However, this movement is substantially restrained by the rotator cuff and/or the shoulder bursa.

In a condition of large rotator cuff tears or rotator cuff arthropathy, as illustrated in FIGS. 3A and 3B, the humerus H may move upwardly along or with respect to the glenoid. The upward movement of the humerus may result in the humeral component HC contacting the clavicle and/or acromion A portion of the scapula S (see FIG. 1). This contact generally proves to be painful to the wearer of the implant, prevents the patient from effectively elevating the humeral shaft, and can, especially as result of repeated contact, result in damage to the acromion and/or clavicle. In addition, movement of the humeral component in and out of proper position relative to a glenoid component, and associated forces applied to the glenoid component, often causes premature loosening of the glenoid component.

U.S. Pat. No. 4,045,826 discloses a shoulder prosthesis including a glenoid component configured as a hemispherical socket. The socket is configured to tightly engage a generally spherical humeral component head. When connected to the scapula, the face of the glenoid component lies in a vertical plane, with the socket located along a generally horizontally extending axis.

This configuration of components has some effectiveness in limiting the vertical movement of the humerus, and thus contact of the humeral component with the clavicle and acromion. Affixing the relatively constrained socket to the acromion of the scapula or the clavicle prevents excessive stresses on the prosthesis-glenoid interface, which is known to cause premature loosening of the glenoid in more constrained glenoid designs used in the past. This is because the ball of the humeral component so tightly fits into the socket of the glenoid component that only a swiveling or rotating motion is generally permitted between those components. On the other hand, this “locking” of the components also substantially reduces the possible range of motion of the prosthesis wearer's arm at the shoulder. As indicated above, normally some vertical movement of the humerus relative to the glenoid is permitted, as is horizontal movement. This prior art prosthesis prevents or limits such movement. Even if the glenoid socket were oversized relative to the humeral head to allow for some translation of the ball within the socket with motion of the shoulder, such a configuration would still prevent any significant upward movement of the humeral head on the glenoid to allow the deltoid muscle to act more efficiently in elevating the arm around the ball and socket fulcrum of the artificial shoulder joint.

Other attempts have been made to “hood” the glenoid component so that it defines a somewhat horizontally extending surface for limiting vertical travel of the humerus. This, however, has only accentuated the above-referenced problems. Such hooded glenoids have been found to be extremely susceptible to premature loosening because of moment forces applied to the unsupported superior hood of the glenoid component.

### SUMMARY OF THE INVENTION

The invention comprises a method and apparatus for total shoulder arthroplasty.

One embodiment of the invention is shoulder prosthesis, including individual prosthesis components. The shoulder prosthesis comprises a humeral component and a glenoid component. The humeral component preferably includes an anchor portion and a head portion. The anchor portion is designed to engage the resected proximal end of a humerus. In one embodiment, the anchor is a tapered body. The head is connected to the anchor and extends there from, preferably at an angle relative to a centerline along which the anchor extends. In one embodiment, the head and anchor are connected by a neck. The head preferably includes a generally cylindrical extension portion and a generally hemispherical portion extending from the cylindrical portion.

The glenoid component is defined by a body having an inner surface and outer surface. At least a portion of the inner surface defines or is part of a concave portion of the body configured to accept at least a portion of the head of the humeral component. In one embodiment, an insert is connected to the inner surface of the glenoid component, the insert defining a head-engaging surface of the glenoid component. The glenoid component is preferably configured to not only anchor to the scapula, but be located adjacent the clavicle and acromion, so as to shield the clavicle and acromion. In one embodiment, the glenoid component may actually contact the clavicle and/or acromion for support, such as by direct anchor or connect thereto. In contrast to the hooded glenoids described in prior art, the superior extension of the glenoid component is supported by the acromion or clavicle, reducing moments on the glenoid component which would otherwise lead to premature loosening.

The outer surface of the body of the glenoid component preferably comprises or is defined by one or more areas of tissue in-growing material. At least a portion of the outer surface which is configured to be located adjacent the glenoid of a patient comprises such a material, for accepting tissue and bone in-growth in that area.

The cavity of the glenoid component is preferably large enough to permit rotational movement of the humeral head in the glenoid cavity. It may be enlarged slightly to allow some limited vertical and lateral movement of the head of the humeral component relative thereto. Preferably, however, at least a portion of the body of the glenoid component defines a stop which limits upward movement of the head of the humeral component, preventing contact of the humeral component with the clavicle or acromion of a patient when the implant is in place.

Another embodiment of the invention is a method of total shoulder arthroplasty. Preferably, the method involves placement and use of the implants of the invention.

In accordance with the method, the proximal end of the humerus is resected, removing the humeral head. The humeral component is affixed to the humerus by locating the anchor portion of the humeral component in the humerus.

The glenoid portion of the scapula of the patient is shaped to fit the outer part of the glenoid prosthesis, which may come in multiple sizes to allow better fitting to each patient's anatomy. The glenoid component is temporarily anchored to the patient in a position such that at least one of the tissue in-growing areas of the glenoid component is located adjacent the resected glenoid, and so that the acromion and scapula of the patient are shielded by the body of the glenoid component. The head portion of the humeral component is then moved into engagement with the cavity of the glenoid component. Permanent anchoring of the glenoid component is realized by tissue and/or bone in growth into the tissue in-growing areas of the component.

In accordance with the invention, a prosthetic shoulder implant is provided which permits translation and rotation of the humeral component relative to the glenoid component, but which at the same time sufficiently limits relative movement of the components to prevent the humeral component from moving into contact with the acromion or clavicle. In this manner, the implant effectively acts as a replacement rotator cuff, such as in the case where total shoulder arthroplasty is needed because of cuff tear arthropathy.

In addition, effective anchoring of the glenoid component is realized, promoting implant longevity. Permanent anchoring is effected by tissue and bone in-growth directly into the glenoid component. Further, in one embodiment, the glenoid component is configured to be supported at an outer or peripheral portion thereof by the acromion or clavicle, providing superior external support of the component.

Further objects, features, and advantages of the present invention over the prior art will become apparent from the detailed description of the drawings which follows, when considered with the attached figures.

### DESCRIPTION OF THE DRAWINGS

FIG. 1 illustrates the normal anatomy of the shoulder;

FIG. 2 illustrates a prior art configuration of shoulder prosthesis;

FIGS. 3A and 3B illustrate undesirable movement of a humeral component of a shoulder prosthesis of the prior art;

FIG. 4 illustrates a glenoid component and a humeral component of a shoulder prosthesis in accordance with the present invention; and

FIG. 5 illustrates an assembled prosthesis of the present invention.

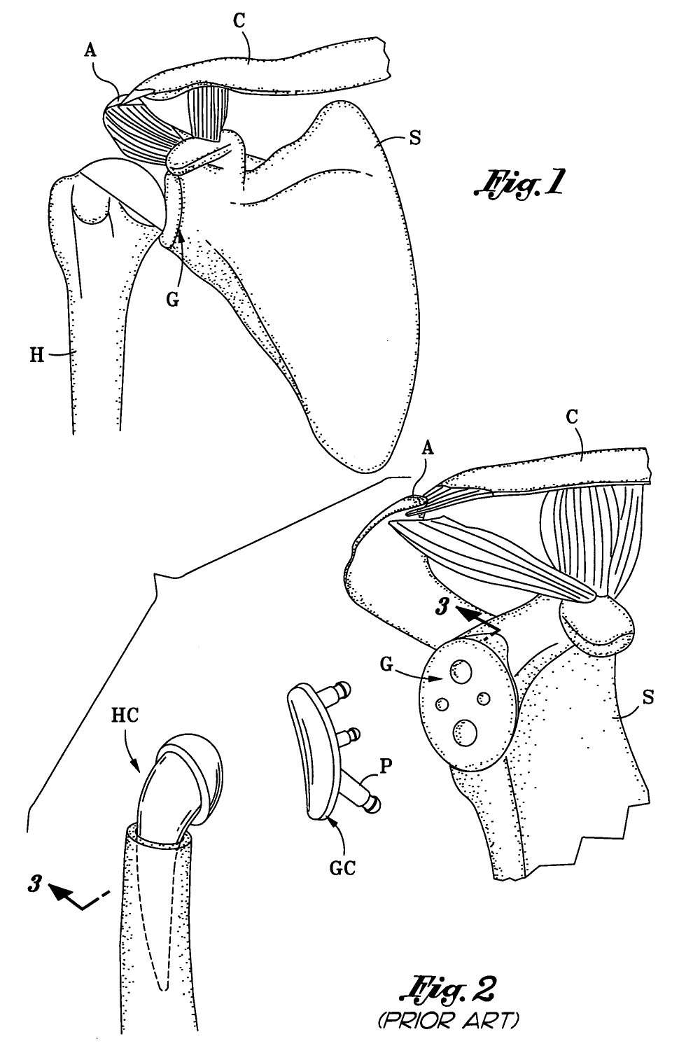

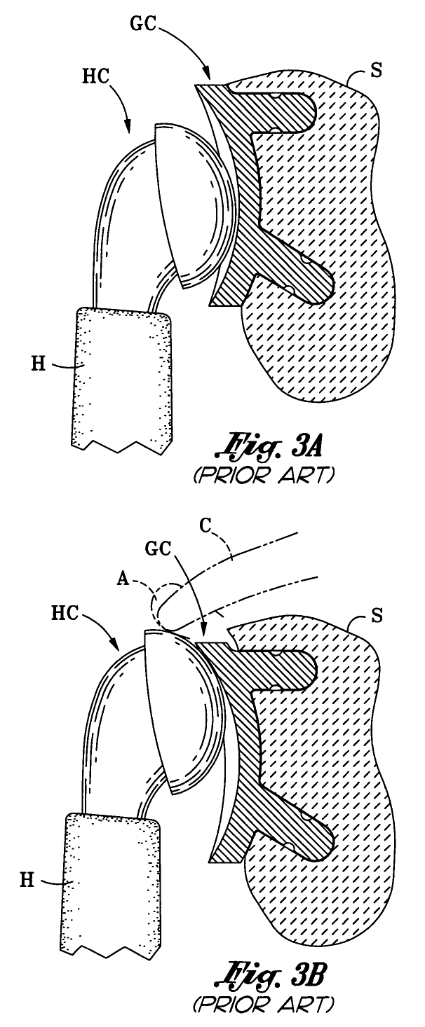

### DETAILED DESCRIPTION OF THE INVENTION

The invention is a method of total shoulder arthroplasty and shoulder arthroplasty prosthesis. In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In general, the invention comprises a shoulder prosthesis, including individual components of the prosthesis, and a method of performing a total shoulder arthroplasty. The components and method are particularly effective in the event shoulder arthroplasty is necessitated or includes a condition of loss of rotator cuff function and rotator cuff arthropathy.

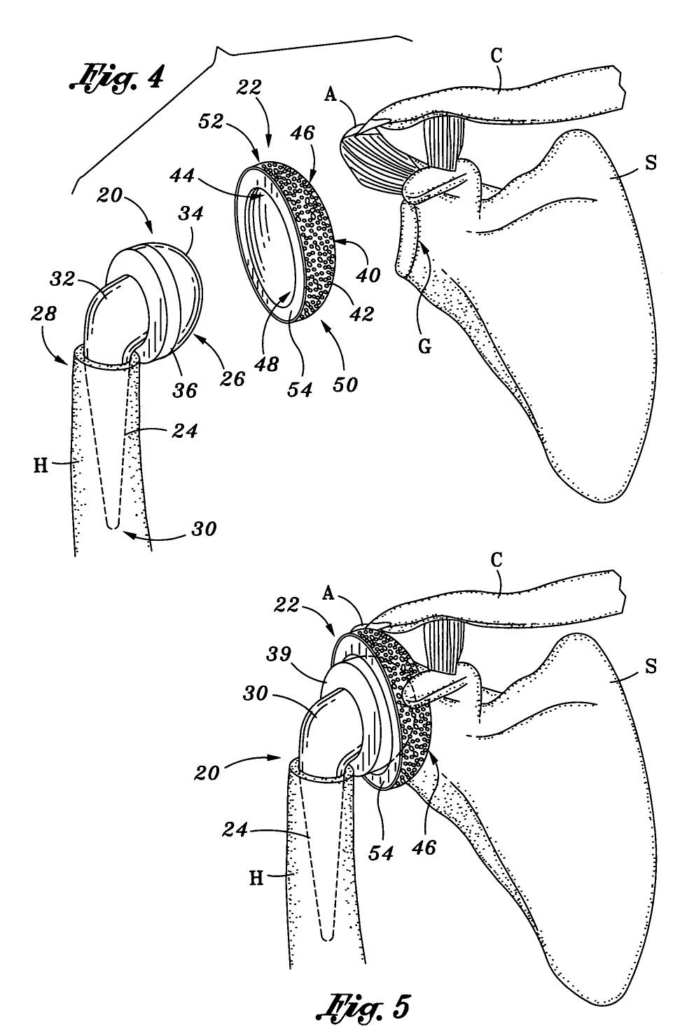

FIG. 4 illustrates one embodiment of a humeral component 20 and a glenoid component 22 of a shoulder prosthesis in accordance with the invention. The humeral component 20 comprises a body defining an anchor 24 and a glenoid component engaging head 26. As illustrated, the anchor 24 is an elongate spike-like element having a top portion or end 28 and a bottom portion or end 30. In one embodiment, the anchor 24 has a generally circular cross-sectional shape. The diameter of the anchor 24 varies from its top end 28 to its bottom end 30, however. As illustrated, the anchor 24 is tapered, with its diameter generally constantly reducing moving from the top end 28 to the bottom end 30.

The exact dimensions of the anchor 24 of the humeral component 20 may vary. For example, the total length of the anchor 24 and/or the diameter of the anchor, may vary. In general, the anchor 24 is configured to fit within the interior space of the humerus and engage the humerus, thus securely connecting the humeral component 20 to the humerus.

The engaging head 26 is preferably connected to the anchor 24. As illustrated, the engaging head 26 is connected to the anchor 24 via a neck 32. As indicated, the anchor 24 is configured to engage the humerus. This requires that the anchor 24 generally extend along the axis of the humerus or parallel thereto.

The engaging head 26 may be any portion of a relatively spherical object, form a small section of such sphere to the near entirety of a sphere extending over the top of the anchor. The engaging head 26 is configured to engage the glenoid component 22, which component serves as a replacement to the normal glenoid surface defined by the scapula. Due to the orientation of the glenoid component 22, which is best illustrated in FIG. 5, this requires that the engaging head 26 be offset from the axis along which the anchor 24 extends. In one embodiment, the neck 32 provides this offset or angular deviation. As illustrated, the neck 32 extends from the top end 28 of the anchor 24 along a line or axis which is offset from the axis along which the anchor 24 extends.

In one embodiment, the neck 32 is a generally cylindrical body. The neck 32 may have approximately the same diameter or outer dimension as the top portion 28 of the anchor 24. In one embodiment, not shown, the neck 32 may define a stop or collar. The stop or collar may be positioned at the interface of the neck 32 and anchor 24 and preferably comprises an outwardly extending body positioned in a plane which is generally perpendicular to the axis along which the anchor 24 extends. This stop or collar may be used to limit the travel of the humeral component 20 into the humerus, including acting as a stop to define a set or installation depth when the anchor 24 is driven into the humerus.

As illustrated, the head 26 extends from the neck 32. Preferably, the head 26 defines a glenoid engaging surface 34. In one embodiment, the head 26 is defined by a body 36 having a first end at which the head 26 is connected to the neck 32, and a second end which defines the glenoid engaging surface 34. At the first, end, the body 36 may be generally cylindrical in shape, and have a diameter which is greater than that of the neck. The body 36 at the second end is preferably generally ball or hemispherical in shape such that the glenoid engaging surface 34 is a generally hemispherical shaped surface.

Preferably, the humeral component 20 is constructed of a strong, durable, bio-compatible, sterilizable material, such as stainless steel, cobalt chrome, or titanium alloy. It is particularly desirable for the material which defines the glenoid engaging surface 34 to be of a material which is highly wear resistant, as the glenoid engaging surface 34 is intended to and will, when the humeral component 20 is utilized as a prosthesis, contact the glenoid component 22.

The humeral component 20 has been described as comprising a plurality of portions or components. It will be appreciated that the humeral component 20 may be constructed as a unitary element, such as in a molding process or by machining from a single block of material. It is also possible for the humeral component 20 to comprise one or more components or elements which are formed independently and then connected to one another, either permanently or semi-permanently, such as by welding, press-fitting together, using bolts or screws, or joining by a taper junction.

The glenoid component 22 will now also be described with reference to FIGS. 4 and 5. Referring first to FIG. 4, the glenoid component 22 comprises a body 40 which defines a bone anchor 42 and a humeral component engaging portion 44.

In a preferred embodiment, the glenoid component 22 is generally half “clam”-shell shaped, having a convex top or outer surface 46 and a concave bottom or inner surface 48. The bone anchor 42 is preferably defined by the convex top 46, while the humeral head engaging portion 44 is defined by the concave bottom 48.

The glenoid component 22 generally has a first or posterior end 50 and a second or superior end 52. The first end or area 50 is configured to connect to, and thus be located adjacent, the scapula S of a patient in the area of their glenoid G. The second end or area 52 of the glenoid component 22 is configured to be located adjacent the clavicle C and acromion A of the patient. As illustrated, in a side view, the top 46 of the glenoid component 22 preferably defines an arcuate or curved surface between the first and second ends 50,52. In the orientation in FIGS. 4 and 5, the elevation of the top 46 increases from the second end 52 towards the first end 50. In one embodiment, the elevation rises quickly, and then remains relatively constant from a middle portion of the component 22.

Preferably, the glenoid component 22 has a depth or width, extending from a front to a rear portion of the component (i.e. generally transverse to the dimension from end to end of the component). This depth is preferably sufficient, as detailed below, for the glenoid component to accept the head 26 of the humeral component 20. The glenoid component 22 may be hemispherical in shape, or it may be truncated anteriorly and posteriorly so that it is taller than it is wide in the sagittal (antero-posterior) plane to allow better fit to the glenoid, acromion, and clavicle, and also to allow better fit within the soft tissue sleeve of the shoulder joint. The top or outer portion 46 of the glenoid component 22 may follow a single radius of curvature, or it may be built up or thicker in one aspect than another, most preferably in the superior portion 50 so as to allow the center of the interior (concavity) of the glenoid to be translated inferiorly or further away from the under surface of the acromion and clavicle.

Preferably, as best illustrated in FIG. 5, the humeral head engaging portion 44 is configured to accept the head 26 of the humeral component 20, and thus comprises a recessed or inset area (i.e. a “socket”) for the head 26. In a preferred embodiment, the glenoid component 22 comprises body defining a humeral head engaging portion 44 configured to accept an insert 54 which defines the inset or socket for accepting the head 26 of the humeral component 20 (thus necessitating that the glenoid component 22 be sized large enough to accept the insert 54). The insert 54 (or the inner surface of the glenoid component 22, if there is no insert) may comprise, for example, a plastic such as an ultra high molecular weight polyethylene. The insert 54 (or inner surface of the glenoid component 22) might instead be manufactured from a metal such as a cobalt chrome alloy, or of a ceramic material

The insert 54 may be snap fit or otherwise connected to the body of the glenoid component 22. In such event, the insert 54 or liner defines the humeral component 20 head engaging-surface. The insert 54 may be symmetrical about a center of rotation (with the head 26 of the humeral component 20), or it may be asymmetric such that the center of rotation of the insert 54 is offset with reference to the center of the body of the glenoid component 22, such that the center of rotation of the glenoid component 22 and head 26 of the humeral component 20 can be offset from the location of the center of the glenoid outer shell. Most commonly, the center of rotation might be located inferior to the center of the body of the glenoid component 22 to push the head 26 of the humeral component 20 inferiorly in order to keep the center of rotation of the head 26 at the level of the patient's true glenoid.

Preferably, the inset or socket portion of the glenoid component 22 (i.e. either as defined by the insert 54 or, if the component 22 does not include an insert, the interior of the glenoid component itself) is substantially the same size or diameter as the head 26 of the humeral component. Or, it may be slightly larger than the head 26 of the humeral component 20, whereby the head 26 may not only rotate, but may translate, relative to the glenoid component 22. However, the head 26 will be relatively constrained in the glenoid concavity such that it cannot translate significantly in the absence of stabilizing rotator cuff muscles. In one embodiment, the insert or socket is thus approximately 1 to no more than about 1.2 times the size (such as diameter) of the head 26 of the humeral component 20 (in the case where the glenoid component 22 is concave, as shown, the distance between the first and second ends 50,52, at the interior surface 48, is about 1-1.2 times or greater the diameter of the head 26). In the case in which the inset or socket is larger than the head 26, the radius of curvature of the humeral head and socket may be substantially the same, or the radius of the socket may be slightly larger than that of the head 26. Preferably, the general size and conformity of the head 26 of the humeral component 20 to the inset or socket of the glenoid component 22 is that it prevents significant superior or anterior/posterior translation of the head 26 relative to the glenoid component 22 during active shoulder motion (but still allow for the intact deltoid muscle to function more efficiently in elevating the shoulder, even if the rotator cuff muscle is deficient).

In an embodiment in which the glenoid component 22 comprises a body configured to accept an insert 54, the interior or bottom portion of the body of the glenoid component 22 preferably still defines an inset for accepting the insert 54, but that inset need not be hemispherical. For example, the body of the glenoid component 22 could define a relatively oval or cube-shaped inset for accepting an insert 54 having a mating exterior shape (such as for additionally reducing the probability of rotation of the insert 54 relative to the body of the glenoid component 22).

In a preferred embodiment, the glenoid component 22 not only defines a humeral head engaging surface 44 which simulates a glenoid, but defines a humeral head 26 travel limiting stop or delimiter. The stop portion of the humeral head engaging surface 44 preferably comprises a superior portion of the glenoid component 22, thus being located towards the second end 52 of the component 22, where the component is located adjacent the clavicle C and acromion A of the patient. In one embodiment, the stop or superior portion comprises an outwardly extending portion of the glenoid component 22 (which portion may be defined as a result of the natural concavity of the glenoid component 22, or by an outwardly extending wing or extension of the component). As illustrated, the glenoid component 22, such as the stop portion, preferably extends outwardly sufficiently to prevent contact by the head 26 of the humeral component 20 with the acromion A or other portion of the patient, when the head 26 is moved upwardly or is rotated.

In a preferred embodiment of the invention, the anchor 42 of the glenoid component 22 comprises one or more areas of bone or tissue in-growing friendly material. In one embodiment, one or more areas of the top or outer surface 46 of the glenoid component 22 are defined by or comprise one or more areas of such material. One such material is Trabecular Metal by Zimmer, Inc. of Warsaw, Ind. Other porous metals, trabecular or otherwise in structure, are now also available. Biologic coatings to enhance bony osseointegration to the component are also available. One such material is hydroxyapatite coating. In a preferred embodiment, only the portions of the top 46 where the glenoid component 22 is, when associated with a patient, located adjacent their scapula S in the area of the glenoid, and/or located adjacent the acromion, comprise such a material. In one embodiment, a layer of such material may extend over a base supporting layer. In another embodiment, areas of tissue in-growing material may be located within, such as inset into, another material.

Aside from the portions of the glenoid component 22 which are, in one embodiment, tissue in-growth friendly, the other portions of the glenoid component 22 may (as detailed above) comprise a high strength, durable, bio-compatible and sterilizable material such as stainless steel, cobalt chrome, titanium, or alloys of such materials. In one embodiment, the majority of the glenoid component 22 is constructed from stainless steel, such as in a molding process. Insets or inlays of the tissue in-growth friendly material may be connected to the main body of the glenoid component 22 or may be formed therewith.

The dimensions of the glenoid component 22 may vary. For example, varying sized components 22 may be constructed for use with patients of differing sizes.

A method of placing the prosthesis components and method of using those components will now be described with reference primarily to FIG. 5. In one embodiment, the shoulder is initially prepared for placement of the components in accordance with known methods of shoulder arthroplasty, in that access is provided to the joint and diseased tissue and other material is removed.

The proximal end of the humerus is resected and prepared for placement of the humeral component 20. Proper determination is made as to the portion of the humeral head which must be removed in order to accommodate the humeral component 20 and effect proper location of the head 26 of that component to the glenoid component 22.

To effect placement of the humeral component 20, the anchor 24 thereof is inserted into the humerus H. Adhesive or other material, such as polymethylmethacrylate cement, may be used to affix the anchor 24 to the humerus H. Or, similar to the glenoid component, the humeral component 20 may have a coating of a porous metal with or without a biologic coating to encourage bony attachment and osseointegration to the humeral component without the use of bone cement, or the component may be cemented in place.

The glenoid component 22 is placed for connection to the scapula S of the patient. In one embodiment, the surface of the scapula is prepared and the glenoid component 22 is positioned so that its first end 50 is aligned for mating with the scapular glenoid and the second end 52 is located adjacent the scapular acromion of the patient. As indicated in more detail below, the glenoid component 22 may initially be anchored with screws, pegs, bolts, wires or the like. Permanent anchoring is, in the preferred embodiment, accomplished by tissue and bone in-growth into the tissue in-growing areas of the component 22.

In one embodiment, the shell or body portion of the glenoid component 22 may be positioned and anchored, and then the insert or liner connected thereto.

Various alternate embodiments of the invention are contemplated. As indicated, in one embodiment, the glenoid component 22 defines a bone anchor 42 configured to anchor the glenoid component to the scapula S, and more particularly the glenoid portion G thereof. In the preferred embodiment, the glenoid component 22 is additionally supported by contact (but not connection to) the acromion A and/or clavicle C.

In another embodiment, however, the glenoid component 22 may also be configured to anchor or connect to the clavicle C and/or acromion A. In the preferred embodiment, the anchor 42 comprises bone-ingrowing material areas with or without a biologic coating of the component. In an alternate embodiment, the anchor 42 may comprise one or more other anchor elements for anchoring to either or both scapula S and clavicle C and/or acromion A. For example, the anchor 42 might comprise one or more pins, pegs, screws, bolts, or other elements which extend from the glenoid component 22 for attachment to the scapula S and/or clavicle C and/or acromion A. Other anchor elements may include screws, metal wires, ties or the like. In a preferred embodiment, such other anchoring elements and methods are employed and provide early fixation (primary stability) until bone and tissue growth is sufficient to permanently anchor the glenoid component 22 (secondary stability).

Numerous features and advantages of the prosthesis components and methods of the invention will now be described in additional detail.

One aspect of the invention is a glenoid component which has an area or portion which substitutes for the glenoid, and a stop or limiter which serves to restrain movement of the humeral component and thus act, some respects, as a rotator cuff. Another aspect of the invention is a glenoid component which is configured to engage the acromion of the patient.

One advantage of the prosthesis of the invention is that the glenoid component 22 defines a humeral head engaging surface 44 which is sufficiently large and open to permit some translation of the head 26 of the humeral component 20 relative thereto, along with rotational movement. This permits the patient's arm to achieve a full range of motion, unlike some prior art prosthesis.

While permitting the wide-ranging normal movement of the patient's arm, the prosthesis is effective in controlling undesirable movement of the humeral component, such as may occur in the event of massive rotator cuff tears or cuff-tear arthropathy. In particular, in a preferred embodiment, a portion of the glenoid component 22 is positioned generally over the head 26 of the humeral component 20 when it is properly positioned. In other words, at least a portion of the glenoid component 22 extends between, and thus shields, the humeral component and the clavicle C and acromion A. As a result, upward travel of the humeral component 20, such as during arm movement, is permitted, but at the same time, upward translation of the fulcrum (or center of rotation) of the humeral component 20, is limited, ensuring proper arm motion. In addition, the interposition of the glenoid component 22 over the head 26 of the humeral component 20, between the humeral component and the clavicle C and/or acromion A protects the clavicle/acromion, and adjacent anatomy from damage which might result if the head 26 were otherwise permitted to travel out of position and contact them.

In one embodiment, the interior or “humeral head” engaging surface of the glenoid component is concave. This has the advantage that a stop of the glenoid component may simply comprise the hood or outwardly extending portion of the glenoid component. Of course, in other embodiments, the stop might comprise a separate element or portion of the glenoid component (rather than simply being a portion which also defines the concavity of the glenoid component).

A particular advantage of the invention is the manner by which the glenoid component 22 is supported. As indicated, the glenoid component 22 is configured to extend over the head 26 of the humeral component 20. The glenoid component 22 thus extends some distance from the scapula. Secure anchoring of the glenoid component 22 to the scapula by tissue and/or bone in-growth ensures that the glenoid component 22 is not loosened as a result of force which may be applied to the glenoid component 22 at varying areas along the component 20. In one embodiment, additional support of the glenoid component 22 is provided by contact of one or more outer or peripheral portions of the glenoid component 22, preferably with the acromion and/or clavicle preventing toggling or “rocking horse” motion of the glenoid when contacted by the humeral head. In one embodiment, this support may include direct bone or tissue in-growth in these areas. The secure anchoring or support at multiple points, including near the first and second ends thereof, counter-acts any “lever” effect and high moment forces which might result. This support and anchoring thus prevents detrimental “rocking” which occurs with prior art component designs.

It will be understood that the above described arrangements of apparatus and the method there from are merely illustrative of applications of the principles of this invention and many other embodiments and modifications may be made without departing from the spirit and scope of the invention as defined in the claims.

What is claimed is:

1. A prosthetic shoulder implant comprising:
a humeral component, said humeral component having an elongate anchor portion with a first end and a second end, said first end configured to be located within a humerus and said second end having a head connected thereto, said head including a first generally cylindrical portion and a second generally hemispherical portion extending from said cylindrical portion; and
a glenoid component, said glenoid component defined by a body having an inner surface and an outer surface, said inner surface defining a concavity of said body for accepting at least the hemispherical portion of said head of said humeral component, one or more portions of said outer surface of said body of said glenoid component comprising a tissue in-growing material.

2. The implant in accordance with claim 1 wherein said glenoid component defines a stop preventing superior migration of said humeral component against an acromion and clavicle of a patient.

3. The implant in accordance with claim 1 wherein said anchor portion of said humeral component is generally spike shaped.

4. The implant in accordance with claim 1 wherein said concavity of said glenoid component has a depth greater than a depth of said humeral component.

5. The implant in accordance with claim 1 wherein said body of said glenoid component has a top end for location adjacent an acromion of a person, said glenoid component having a stop at said top end.

6. A method of shoulder arthroplasty of a shoulder of a patient comprising the steps of:
resecting a proximal end of a humerus of said patient to remove a humeral head portion thereof;
providing a humeral component of a prosthetic shoulder implant, said humeral component having an elongate anchor portion with a first end and a second end and a head extend from said anchor;
locating at least a portion of the anchor portion of said humeral component in said resected humerus;
resecting a glenoid portion of a scapula of said patient;
providing a glenoid component of said prosthetic shoulder implant, said glenoid component defined by a body having an inner surface and an outer surface, said inner surface defining a humeral head-accepting concavity, one or more portions of said outer surface of said body comprising tissue-in-growing material;
temporarily anchoring said glenoid component so that at least one tissue in-growing material area is located adjacent said resected area of said glenoid portion of said scapula; and
allowing tissue to ingrow said tissue in-growing material to permanently anchor said glenoid component to said scapula of said patient.

7. The method in accordance with claim 6 further comprising the step of location at least a portion of said head of said humeral component within said concavity of said glenoid component.

8. The method in accordance with claim 6 wherein said glenoid component defines a stop preventing superior migration of said head of said humeral component against an undersurface of an acromion and a distal clavicle of said patient, and further comprising the step of orienting said glenoid component so that said stop is located adjacent said acromion of a patient.

9. The method in accordance with claim 6 further comprising the step of locating at least a portion of said glenoid component beneath an acromion of said patient and temporarily attaching said glenoid component to an undersurface of said acromion and permanently affixing said glenoid component to said acromion via ingrowth to said outer surface of said body of said glenoid component.

10. The method in accordance with claim 6 further comprising the step of locating at least a portion of said glenoid component between a clavicle and acromion of said patient and said head of said humeral component.
